(19) United States
(12) Patent Application Publication
(10) Pub. No.: US 2005/0177084 A1
(43) Pub. Date: Aug. 11, 2005

(54) Diabetic toe protectors

(76) Inventors: Allan L. Green, Tamarac, FL (US); Michael Anthony, Coral Springs, FL (US)

Correspondence Address: Mark D. Bowen;Stearns Weaver Miller, et al., Suite 1900, 200 East Broward Boulevard, Fort Lauderdale, FL, 33301, US

(21) Appl. No.: 10/772,865

(22) Filed: Feb. 6, 2004

(51) Int. Cl. A61F 5/00

(52) U.S. Cl. 602/30

(57) ABSTRACT

A toe protector for protecting the toe and sores or bunions on the foot of a patient from irritation, abrasion, and impact, so as to promote healing is disclosed. The toe protector comprises a generally tubular body sized to substantially cover a toe of the user. The device is preferably fabricated from a soft, FDA approved silicon material, in a double-walled tubular configuration wherein inner and outer layers form an interstitial space. The interstitial space is filled with substance, such as a liquid or gel. A flap extends along the inside portion of the foot and includes a recessed portion sized and positioned for covering relation with bunions on the metatarsophalangeal joint or other sores. According to another aspect, the present invention provides a foot protector to be worn on the foot of a patient. The foot protector preferably incorporates one or more water-filled toe protectors as generally described above. Each of the embodiments may define a plurality of ventilation holes formed by welding of inner and outer portions.

### CROSS REFERENCE TO RELATED APPLICATIONS

N/A

### STATEMENT REGARDING FEDERALLY SPONSORED RESEARCH OR DEVELOPMENT

N/A

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office patent file or records, but otherwise reserves all copyrights.

### BACKGROUND OF THE INVENTION

1. Field of the Invention

The present invention relates generally to a medical protector for use on the foot of a bedridden patient and more particularly, to a toe protector that protects sores and bunions on the feet of a diabetic patient.

2. Description of the Background Art

Many diabetic patients suffer from bunions and sores of the feet. A bunion is an enlargement of the joint at the base of the hallux (“big toe”)—the metatarsophalangeal (MTP) joint—that forms when the bone or tissue at the big toe joint moves out of place. This forces the toe to bend toward the others, causing an often painful lump of bone on the foot. Since this joint carries a substantial amount of body weight while walking, bunions can cause extreme pain if left untreated. The MTP joint itself may become stiff and sore, making even the wearing of shoes difficult or impossible. Bunions—from the Latin “bunio,” meaning enlargement-can also occur on the outside of the foot along the little toe, where it is called a “bunionette” or “tailor's bunion.”

Bunions and sores developed by diabetic patients are often slow and sometimes difficult to heal as a result of complications resulting from diabetes. Without proper treatment and care, bunions and sores are susceptible to infection and other complications that inhibit the healing process. The healing process may be further complicated when the patient is bedridden due to extended contact of the skin with bedding materials.

There exist a number of protectors on the market today intended for use on the foot. These foot protectors range from the inexpensive, non-durable type to the expensive, bulky type that are very heavy and difficult to use or walk on. Further, many of the foot protectors available fail to specifically address bunions and sores on the MTP joint. Accordingly, there exists a need for an improved toe protector for use by patients suffering from sores of the feet, particularly bunions.

### BRIEF SUMMARY OF THE INVENTION

The present invention provides a toe protector to be worn on the foot of a patient to protect sores on the toe from irritation, abrasion, and impact, so as to promote healing. The toe protector comprises a generally tubular body sized to substantially cover a toe of the user. The device is preferably fabricated from a soft, FDA approved silicon material, in a tubular configuration adapted to be filled with water or other suitable liquid. The device preferably includes an inner recessed portion sized and positioned on the device so as to cover bunions on the metatarsophalangeal joint. According to another aspect, the present invention provides a foot protector to be worn on the foot of a patient. The foot protector preferably incorporates one or more water-filled toe protectors as generally described above. Each of the embodiments may define a plurality of ventilation holes formed by welding of inner and outer portions.

Accordingly, it is an object of the present invention to provide an improved toe protector for use by those suffering from sores, bunions, or other abnormalities of the feet.

Still another object of the present invention is to provide a liquid filled toe protector fabricated from a soft silicone material.

In accordance with these and other objects, which will become apparent hereinafter, the instant invention will now be described with particular reference to the accompanying drawings.

### BRIEF DESCRIPTION OF THE SEVERAL VIEWS OF THE DRAWINGS

The drawings furnished herewith illustrate a preferred construction of the present invention in which the above advantages and features are clearly disclosed as well as other which will be readily understood from the following description of the illustrated embodiment.

FIG. 1 is a perspective view of a toe protector according to a preferred embodiment of the present invention in position to be placed on the toe of a user's foot;

FIG. 2 is a perspective view of the toe protector;

FIG. 3 is a side perspective view of the toe protector on the toe of a user;

FIG. 4 is a top perspective view of the toe protector secured to the user's toe by a strap;

FIG. 5 is a perspective view of the toe protector and securing strap;

FIG. 6 is a medial perspective view of the toe protector;

FIG. 7 is a medial perspective view thereof in partial section; and

FIG. 8 depicts a perspective view of an alternate embodiment foot protector according to the present invention.

### DETAILED DESCRIPTION OF THE INVENTION

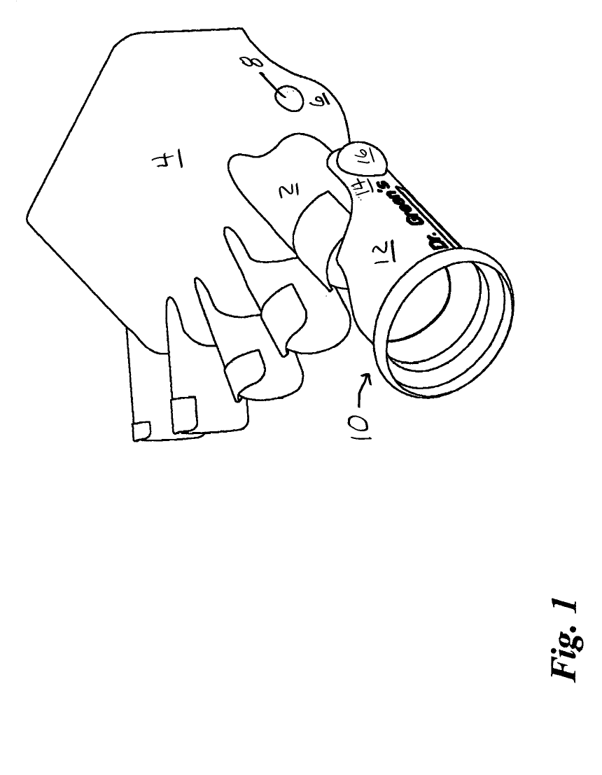

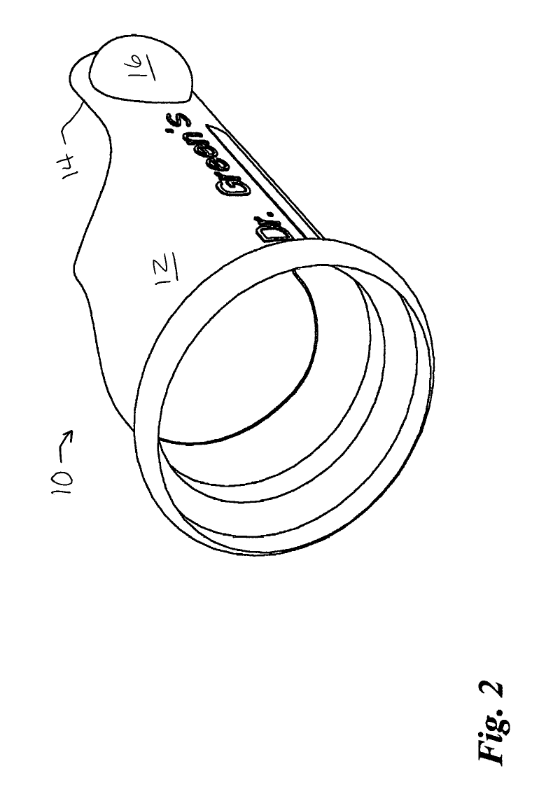

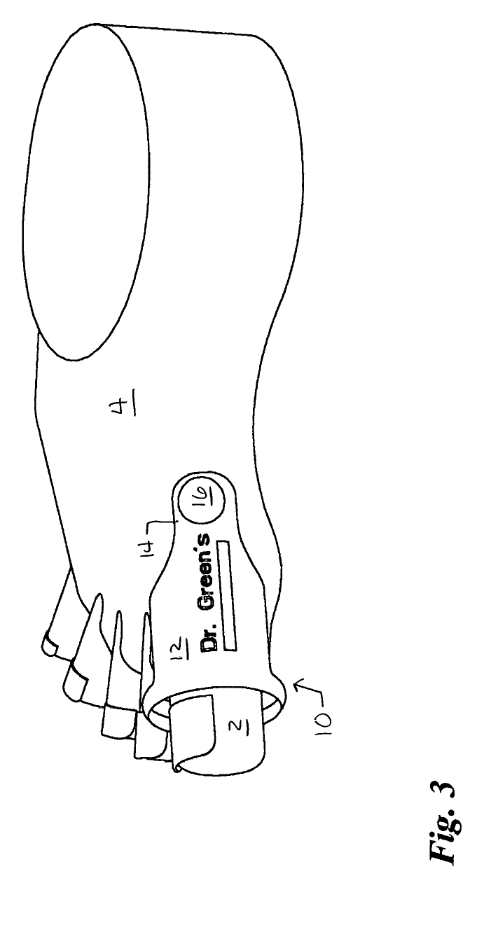

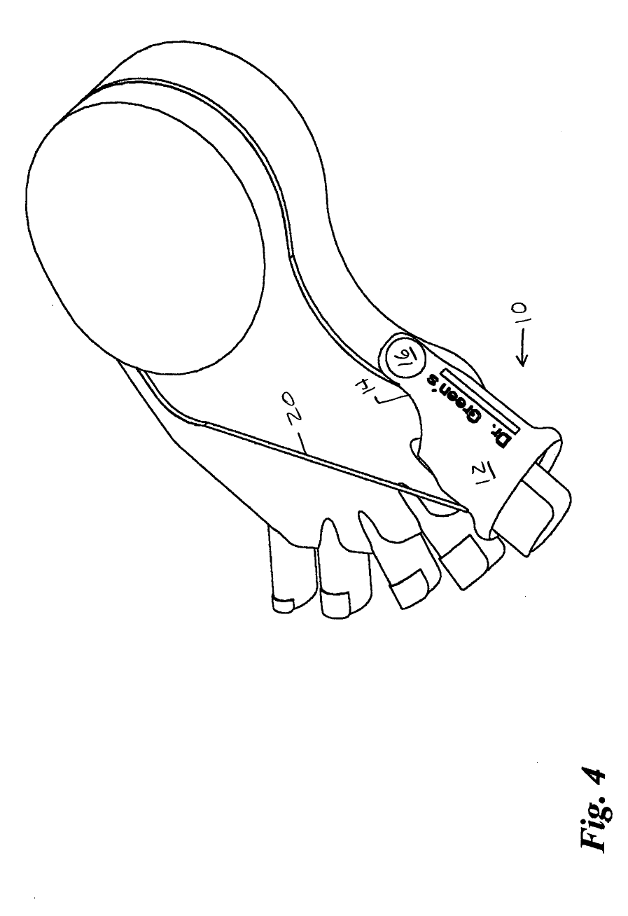

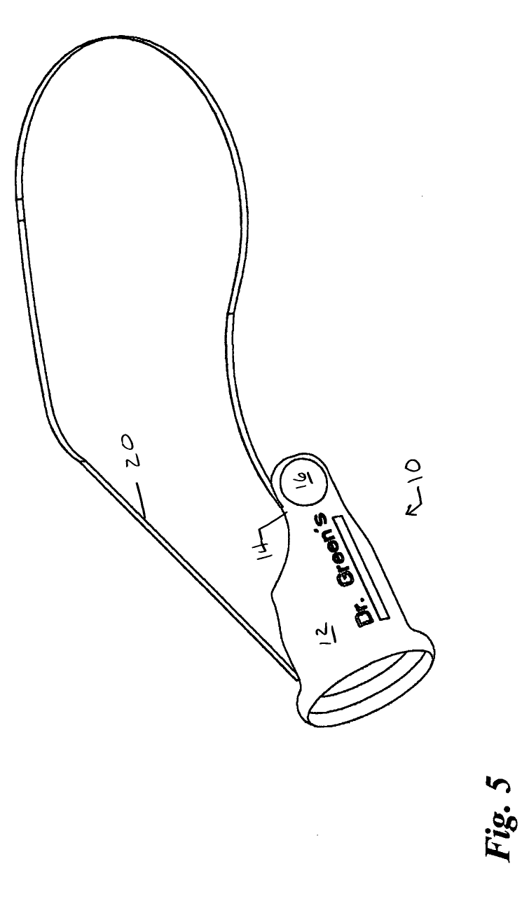

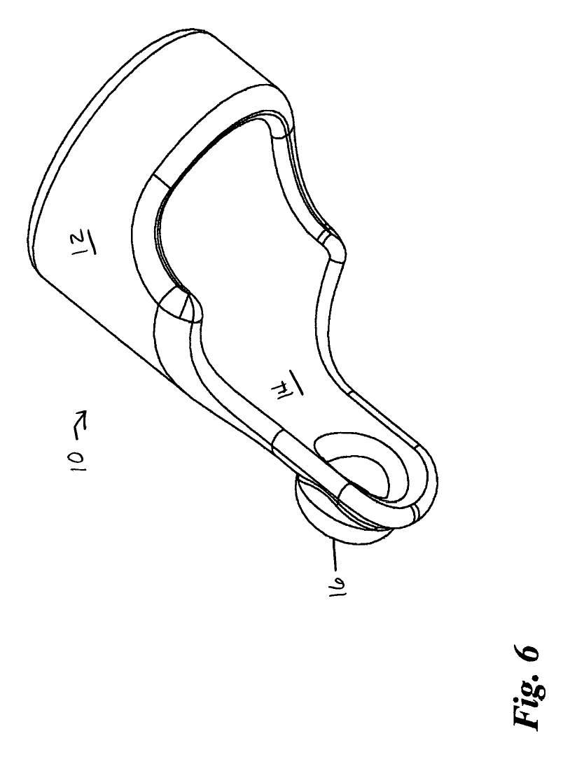

With reference to the drawings, FIGS. 1 through 7 depict a preferred embodiment of a toe protector according to the present invention, generally referenced as 10. Toe protector 10 is preferably adapted to be worn on the big toe so as to substantially cover the big toe and metatarsophalangeal (MTP) joint, where bunions and other sores commonly form. As best depicted in FIGS. 1-3, toe protector 10 comprises a generally tubular body 12 sized to be comfortably worn on the big toe 2 of a user's foot 4. Toe protector 10 includes an elongated projecting lateral side flap 14 having a inner surface defining a recessed cavity 16 adapted to extend over the MTP joint 6 in covering relation with a bunion 8. As best depicted in FIG. 5, toe protector 10 may be adapted with a strap 20 for securing toe protector 10 on the user's foot. FIG. 4 depicts the use of strap 20 to hold toe protector 10 on the user's foot in a first configuration, and FIG. 9 depicts the use of an alternate strap configuration.

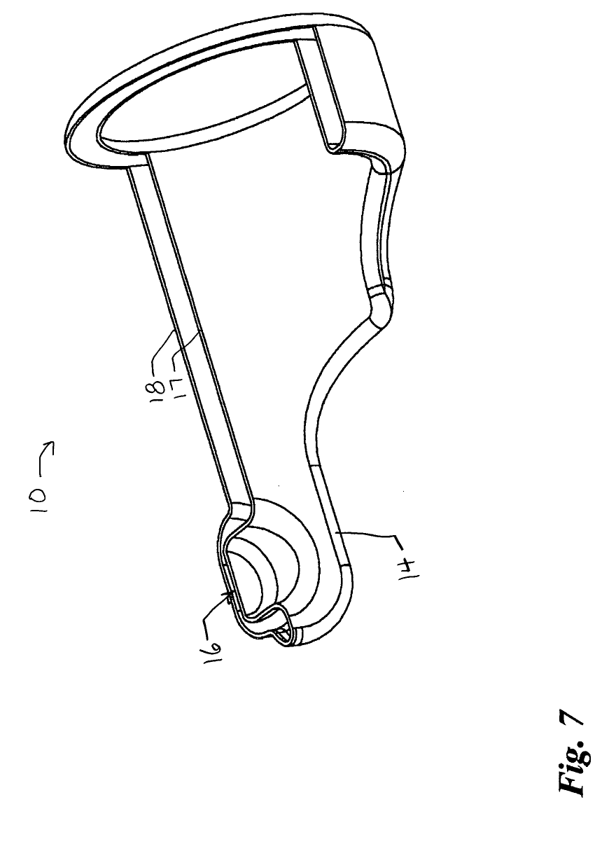

As discussed hereinabove, toe protector 10 comprises a generally tubular body sized to substantially cover a toe of the user. Toe protector 10 is preferably fabricated from a soft, FDA approved silicon material, in a tubular configuration adapted to be filled with water or other suitable liquid. More particularly, as best depicted in FIG. 7, toe protector 10 is preferably fabricated as an open ended tubular structure having inner and outer sleeve members, referenced as 17 and 18 resepctively. Inner and outer sleeve members 17 and 18 are sealingly connected to form a double wall tubular toe protector defining an interior volume. As seen in FIG. 7, inner and outer sleeves 17 and 18 each define a radially inner surface forming a concave recess, generally referenced as 16, on flap 14 that is sized for covering relation with a bunion or sore located on or in proximity to the MTP joint on the user's foot.

As should be apparent, toe protector 10 is preferably sized and shaped to conform to the size and shape of the toe. According to significant aspect of the present invention, toe protector 10, and particularly the interior volume defined between inner and outer sleeves 17 and 18, is preferably filled with a liquid, such as water, a gel substance, or any other suitable filling agent. The water filled toe protector may be cooled in a refrigerator or heated in a microwave oven to provide therapeutic relief as is desirable. When filled, toe protector 10 is inserted over the user's big toe and may be held in place by either particular sizing for a snug fit, or by use of an attached strap 20. By providing a filled toe protector fabricated from a soft, silicone-like material, it has been found that a toe protector 10 having a projecting portion for covering the MTP joint as disclosed herein is effective in protecting the toe, and more significantly for protecting and cushioning bunions and sores on or near the MTP joint. Accordingly, the toe and MTP area are effectively protected from impact and irritating abrasion.

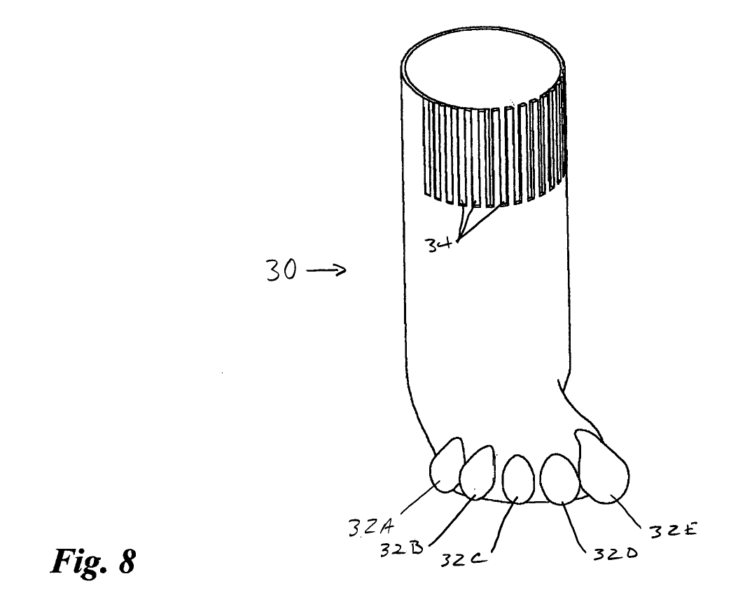

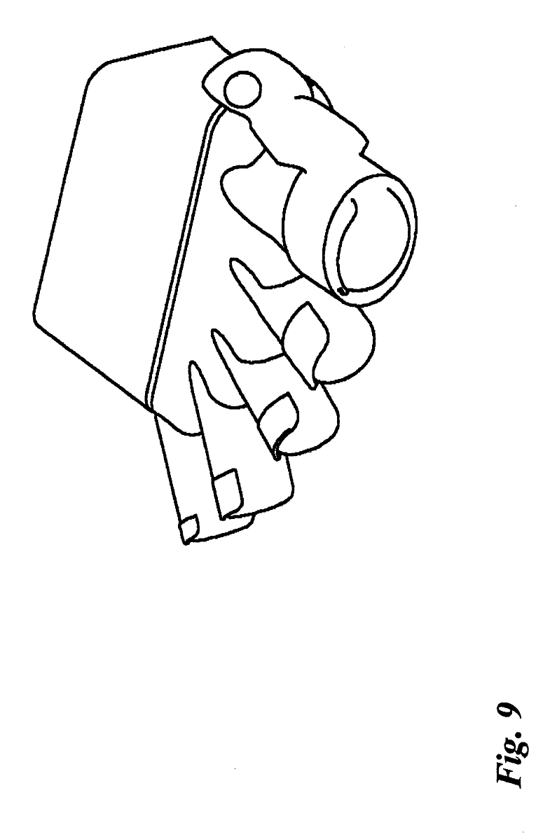

FIG. 8 depicts an alternate embodiment wherein the improvements disclosed herein above relating to toe and foot protection are incorporated into a sock-like foot protecting embodiment, generally referenced as 30. Sock-like foot protector 30 is preferably fabricated with soft padded foam to protect the toes, or may be fabricated using similar medically approved materials, such as silicone, in a water filled double walled structure in the form of a sock having individual toe compartments, referenced as 32A-32E. Various embodiments are contemplated wherein any one toe, combination of toes, or all toes may be adapted with the water-filled cushioned structure. In addition, the embodiment depicted in FIG. 8 discloses air vents 34 formed in the structure for ventilation and comfort. Air vents 34 are preferably formed by welding the inner and outer layers into any suitable sized or shaped ventilation openings. The air vents may be formed in any desirable number, size, or shape, and may be located on the device in any suitable location. As should be apparent, the toe protector embodiment depicted in FIGS. 1-7 may likewise be adapted with one or more air vents.

The instant invention has been shown and described herein in what is considered to be the most practical and preferred embodiment. It is recognized, however, that departures may be made therefrom within the scope of the invention and that obvious structural and/or functional modifications will occur to a person skilled in the art.

1. A toe protector to be worn on the foot of a patient, the toe protector comprising:
a tubular double-walled body wherein radially inner and outer walls define an interstitial space;
a substance filling said interstitial space;
a distal end defining an opening in the body for exposing a distal end portion of the patient's big toe;
a proximal opening in the body for insertion of the patient's big toe;
said body proximal end including an elongate flap for covering at least a portion of the metatarsophalangeal joint.

2. A toe protector according to claim 1, further including a strap for securing said body to the patient's foot.

3. A toe protector according to claim 1, wherein said elongate flap defines a recess defined by a concave surface.

4. A toe protector according to claim 1, further including at least one air vent defined by said double-walled body.

5. A toe protector according to claim 1, wherein said substance is a gel.

6. A toe protector according to claim 1, wherein said substance is liquid.

7. A toe protector according to claim 6, wherein said liquid is water.

8. A toe protector to be worn on the foot of a patient, the toe protector comprising:
a tubular double-walled body wherein radially inner and outer walls define an interstitial space therebetween;
a substance filling said interstitial space;
a distal end defining an opening in the body for exposing a distal end portion of the patient's big toe;
a proximal opening in the body for insertion of the patient's big toe;
said body proximal end including an elongate flap for covering at least a portion of the metatarsophalangeal joint;
said elongate flap having a surface disposed generally adjacent to the patient's foot, said surface having a concave portion disposed in covering relation with a sore on the patient's foot.

9. A toe protector according to claim 8, further including a strap for securing said body to the foot of a patient.

10. A toe protector according to claim 8, wherein said substance comprises a gel.

11. A toe protector according to claim 8, wherein said substance is liquid.

12. A toe protector according to claim 11, wherein said liquid is water.
